(19) United States
(12) Patent Application Publication
(10) Pub. No.: US 2003/0147975 A1
(43) Pub. Date: Aug. 7, 2003

(54) Pharmaceutical composition which produces irritation

(76) Inventors: Yatindra Joshi, Princeton, NJ (US); Russell Somma, Sparta, NJ (US)

Correspondence Address: THOMAS HOXIE, NOVARTIS, CORPORATE INTELLECTUAL PROPERTY, ONE HEALTH PLAZA 430/2, EAST HANOVER, NJ, 07936-1080, US

(21) Appl. No.: 10/385,372

(22) Filed: Mar. 10, 2003

Related U.S. Application Data
(62) Division of application No. 09/942,809, filed on Aug. 30, 2001.

(51) Int. Cl. A61K 35/78; A61K 31/00

(52) U.S. Cl. 424/725.1; 424/725; 424/195.18

(57) ABSTRACT

A pharmaceutical composition which reduces or eliminates the drug abuse potential of central nervous system stimulant comprising: (a) a central nervous system stimulant selected from the group consisting of methylphenidate, amphetamine, methamphetamine, and combinations thereof; and (b) a mucous membrane irritant selected from the group consisting of organic and inorganic acid, salt, ketone, nitrite, sulfide, bisulfate, persulfate, glycerophosphate, hypophosphate, borate, titanate, amino acid, peptide, and combinations thereof, wherein the mucous membrane irritant produces irritation when contacted with the skin or mucous membrane. The present invention is based on the discovery that a central nervous system stimulant, such as methylphenidate, in combination with a mucous membrane irritant, such as citric acid, reduces or eliminates potential drug abuse by producing “irritation” when contacted with the dermis layer of skin or mucous membrane, and thus, prevents nasal absorption and/or injectability of the drug.

### FIELD OF THE INVENTION

The present invention relates to a pharmaceutical composition comprising a central nervous system stimulant such as methylphenidate and a mucous membrane irritant. The composition reduces or eliminates potential drug abuse by producing “irritation” when contacted with the dermis layer of skin or mucous membrane, and thus, prevents nasal absorption and/or injectability of the drug.

### BACKGROUND OF THE INVENTION

Methylphenidate, which is commercially available under the trademark Ritalin® from Novartis Pharmaceuticals Corporation, is generally classified as a central nervous system stimulant. Other examples of drugs which are classified as central nervous stimulants are amphetamine and methamphetamine. Central nervous stimulants are known to activate the brain stem arousal system to effect stimulation of the patient.

Methylphenidate is the most commonly prescribed psychotropic medication for children in the United States, primarily for the treatment of children diagnosed with attention deficit disorder (ADD) and Attention Deficit Hyperactivity Disorder (ADHD), and thus, is widely available. In addition, methylphenidate has been found to be particularly useful for treating Acquired Immunodeficiency Syndrome (AIDS) patients who suffer from cognitive decline. See Navia et al., Annals of Neurology, 19:517-524 (1986).

The use of methylphenidate is described in U.S. Pat. Nos. 2,838,519 and 2,957,880. U.S. Pat. Nos. 5,922,736; 5,908,850; 5,773,478; 6,113,879 describe administering d-threo methylphenidate to treat nervous system disorders. U.S. Pat. Nos. 5,936,091 and 5,965,734 describe processes and intermediates for preparing 2-substituted d-threo piperidines. U.S. Pat. Nos. 6,100,401; 6,121,453; and 6,162,919 describe processes for preparing substantially the single enantiomer d-threo methylphenidate. U.S. Pat. Nos. 5,874,090 and 5,837,284 describe sustained release formulations of methylphenidate.

In addition to important medical uses, central nervous system stimulants are increasingly employed for illicit purposes including emotional, psychological, euphoric, hallucinogenic, and psychedelic experiences, by such means as inhalation and intravenous administration. These purposes and the physical dependence accompanying the administration of these drugs has led to drug abuse. Drug abuse has become for many habituates a way of life. Moreover, to a rapidly growing segment of the world population, use of these drugs is often seen as fashionable.

WO 97/33566 describes an opioid composition which has a low potential for abuse, and is achieved by incorporating an opioid antagonist in the composition in order to reduce the effect of the opioid. Examples of opioid antagonists disclosed in WO 97/33566 are naltrexone, naloxone, nalmefene, nalide, nalmexone, nalorphine, nalpuphine, nalorphine, and dinicotinate.

While central nervous stimulants are a necessary part of modern medicine, it would be highly desirable to provide a pharmaceutical composition comprising a central nervous stimulant which reduces or eliminates drug abuse potential without decreasing the effectiveness of the central nervous stimulant.

### SUMMARY OF THE INVENTION

The present invention relates to a pharmaceutical composition which reduces or eliminates the drug abuse potential of central nervous system stimulant comprising:

(a) a central nervous system stimulant selected from the group consisting of methylphenidate, amphetamine, methamphetamine, and combinations thereof; and

(b) a mucous membrane irritant selected from the group consisting of organic and inorganic acid, salt, ketone, nitrite, sulfide, bisulfate, persulfate, glycerophosphate, hypophosphate, borate, titanate, amino acid, peptide, and combinations thereof, wherein the mucous membrane irritant produces irritation when contacted with the dermis layer of skin or mucous membrane. A preferred mucous membrane irritant is citric acid.

The present invention is based on the discovery that a central nervous system stimulant, such as methylphenidate, in combination with a mucous membrane irritant, reduces or eliminates potential drug abuse by producing “irritation” when contacted with the dermis layer of skin or mucous membrane, and thus, prevents nasal absorption and/or injectability of the drug. Typical symptoms or signs of “irritation” include itching, stinging, burning, tingling, erythema (redness), and edema (swelling).

### DESCRIPTION OF THE INVENTION

The invention is directed to a pharmaceutical composition which reduces or eliminates the drug abuse potential of a central nervous system stimulant. The composition comprises a central nervous system stimulant and a mucous membrane irritant. Component (a) of the composition of the invention is a central nervous system stimulant such as methylphenidate, amphetamine, and methamphetamine. Pharmaceutically acceptable salt forms of the central the nervous system stimulant are included within the term “central nervous system stimulant”. A combination of central nervous system stimulants may also be used.

As used herein, “methylphenidate” includes the following four optical isomers: d-threo-methylphenidate, l-threo-methylphenidate, d-erythro-methylphenidate, and l-erythro-methylphenidate. A preferred isomer is d-threo-methylphenidate. A combination of isomers may also be used, for example, dl-threo-methylphenidate. Most preferably, the methylphenidate is methylphenidate hydrochloride.

The effective dosage for the central nervous system stimulant may vary depending on the concentration of the drug, the mode of administration, the condition being treated, and the severity of the condition being treated. In addition, the effective dosage depends on a variety of factors which are specific to the patient being treated, such as species type, age, weight, and sex.

In a preferred embodiment of the invention, the amount of central nervous system stimulant in the compositions of the invention is from about 0.1 to about 90 weight percent, more preferably from about 1 to about 50 weight percent, based on the total weight of the composition. Most preferably, the amount of central nervous system stimulant in the compositions is from about 2 to about 10 weight percent, based on the total weight of the composition.

Component (b) of the composition of the invention is a mucous membrane irritant. The mucous membrane irritant is any chemical or compound which produces “irritation” including various symptoms or signs, when contacted with the dermis layer of skin or mucous membrane. Typical symptoms or signs of “irritation” include itching, stinging, burning, tingling, erythema (redness), and edema (swelling). The mucous membrane irritant may be used alone or in combination with other mucous membrane irritants. The mucous membrane irritant is selected from organic and inorganic acids, salts, ketones, nitrites, sulfides, bisulfates, persulfates, glycerophosphates, hypophosphates, borates, titanates, amino acids, and peptides.

Specific examples of mucous membrane irritants include, but are not limited to, the following: anthralin, camphor, canthariden, capsicum, coal tar, ichthammol, juniper tar, menthol, Peruvian balsam, pine tar, aluminum chloride, resorcinol, storax, tolu balsam, nitric acid, phenol, podofilox, podophyllum, potassium hydroxide, silver nitrate, trichloroacetic acid, benzoyl peroxide, fluorouracil, salicylic acid, retinoic acid, ethanol, isopropanol, selenium sulfide, benzalkonium chloride, allantoin, aminobenzoic acid, propenoic acid, dihydroxyacetone, dioxybenzone, octyl methoxycinnamate, 2,4,6,8-nonanetetraenoic acid, homosalate, hydrogen peroxide, hydroxyurea, citric acid, lactic acid, glycolic acid, salicylic acid, pyromellitic acid, pyromellitic dianhydride, pyruvic acid, acetic acid, acrylic acid, trichloroacetic acid, 1-pyrrolidone-5-carboxylic acid, capryloyl salicylic acid, hydroxy decanoic acid, hydroxy octanoic acid, gluconolactone, methoxypropyl gluconamide, malic acid, maleic acid, tartaric acid, mandelic acid, gluconic acid, sodium chloride, ethylenediaminetetraacetic acid disodium salt, sodium boroformate, sodium bicarbonate, and dipropyl ketone. In a preferred embodiment the mucous membrane irritant is a mono-, di-, tri-, or tetra-carboxylic acid, more preferably citric acid.

The amount of mucous membrane irritant in the compositions of the invention is preferably from about 0.1 to 60 weight percent, based on the total weight of the composition. More preferably, the amount of mucous membrane irritant is from about 1 to about 40 weight percent, more preferably from about 5 to about 20 weight percent, based on the total weight of the composition.

Additional ingredients which may be used in the compositions of the invention include natural and/or artificial ingredients which are commonly used to prepare oral pharmaceutical dosage forms. Examples of additional ingredients include enteric coating agents, diluents, binders, humectants, disintegrants, anti caking agents, fibers, solubilizers, emulsifiers, flavorants, sweeteners, enzymes, fillers, buffers, stabilizers, colorants, dyes, plasticizing agents, antioxidants, anti-adherents, preservatives, electrolytes, glidants, lubricants, and carrier materials. A combination of additional ingredients may also be used. Such ingredients are known to those skilled in the art, and thus, only a limited number will be specifically referenced. Preferably the additional ingredients are used in the compositions of the invention in an amount that corresponds to an amount generally recognized as safe (GRAS) and effective by the United States Food and Drug Administration, the Environmental Protection Agency, the United States Department of Agriculture, or other comparable regulatory agency. For those additional ingredients for which no regulatory approval has been obtained, then an amount generally accepted in the art as both safe and efficacious is preferred.

Examples of humectants that can be used in the compositions of the invention include but are not limited to: sucrose, sorbitol, glycerol, propylene glycol, poly-(ethylene glycol), N-methyl pyrrolidone, N-ethyl pyrrolidone, diacetone alcohol, gamma.-butyryl lactone, ethyl lactate, low molecular weight polyethylene glycol, or combinations thereof.

Examples of glidants that can be used in the compositions of the invention include but are not limited to: silica, magnesium trisilicate, powdered cellulose, starch, and talc. Colloidal silica and colloidal silicone dioxide are particularly preferred.

Examples of fillers that can be used in the compositions of the invention include but are not limited to: sugar, dextrates, dextrin, dextrose, lactose, sucrose, mannitol, microcrystalline cellulose, powdered cellulose, sorbitol, and tribasic calcium phosphate.

Examples of lubricants that can be used in the compositions of the invention include but are not limited to: stearic acids and its salts such as Mg, Al or Ca stearate, polyethylene glycol 4000-8000, talc, sodium benzoate, sodium acetate, leucine, sodium oleate, sodium lauryl sulfate, and magnesium lauryl sulfate.

Examples of solubilizers and/or emulsifiers that can be used in the compositions of the invention include but are not limited to: sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl)imidazolidone-(2). In this context, polyoxyethylated means that the substances in question contain polyoxyethylene chains, the degree of polymerization of which generally lies between 2 and 40 and in particular between 10 and 20.

Examples of antioxidants that can be used in the compositions of the invention include but are not limited to: sodium sulphite, sodium hydrogen sulphite, sodium metabisulphite, ascorbic acid, ascorbylpalmitate, -myristate, -stearate, gallic acid, gallic acid alkyl ester, butylhydroxyamisol, nordihydroguaiaretic acid, tocopherols as well as synergists (substances which bind heavy metals through complex formation, for example lecithin, ascorbic acid, phosphoric acid ethylene diamine tetracetic acid, citrates, tartrates). Addition of synergists substantially increases the antioxygenic effect of the antioxidants.

Examples of preservatives that can be used in the compositions of the invention include but are not limited to: sorbic acid, p-hydroxybenzoic acid esters, benzoic acid, sodium benzoate, trichloroisobutyl alcohol, phenol, cresol, benzethonium chloride, chlorhexidine and formalin derivatives.

The total amount of additional ingredients in the compositions of the invention are preferably from about 30 to about 75 weight percent, based on the total weight of the composition. More preferably, the total amount of additional ingredients is from about 50 to about 70 weight percent, most preferably from about 53 to about 67 weight percent, based on the total weight of the composition.

The following examples further describe the materials and methods used in carrying out the invention. The examples are not intended to limit the invention in any manner.

### EXAMPLE 1

Preparation of Chewable Tablets Containing 2.5% Methylphenidate and 10% Citric Acid.

Composition

| | |
|---|---|
| dl-methylphenidate | 5.0 gm |
| citric acid | 20.0 gm |
| lactose | 75.0 gm |
| talc | 3.0 gm |
| mannitol | 90.0 gm |
| stearic acid | 2.0 gm |
| 5% gelatin solution in demineralized water | 4.0 gm |
| saccharin | 1.0 gm |

All the solid ingredients are first forced through a sieve of 0.25 mm mesh width. The mannitol, dl-methylphenidate, and lactose are mixed, granulated with the addition of gelatin solution, forced through a sieve of 2 mm mesh width, dried at 50° C. and again forced through a sieve of 1.7 mm mesh width. Citric acid, talc and saccharin are added to the dried mixture of drug substance. The stearic acid is added and the final blend is made. The resulting blend is compressed to form 7 mm round standard concave tablets.

### EXAMPLE 2

Preparation of Tablets Containing 4% Methylphenidate and 16% Citric Acid.

Composition

| | | |
|---|---|---|
| d-methylphenidate | 10.0 | gm |
| PEG 8000 | 3.0 | gm |
| sucrose | 3.0 | gm |
| starch | 20.0 | gm |
| lactose | 170 | gm |
| talc | 2.0 | gm |
| magnesium stearate | 2.0 | gm |
| citric acid | 40.0 | gm |
| demineralized water | | |

All the solid ingredients are first forced through a sieve of 0.6 mm mesh width. The dl-methylphenidate, a portion of the lactose, starch, and sucrose are mixed then granulated with the PEG 8000 solution. The granulation is dried overnight at 50° C., and then forced through a sieve of 1.2 mm mesh width. The remaining lactose, talc, magnesium stearate and citric acid are blended with the dried material. The resulting blend is compressed to form 8 mm round standard concave tablets.

### EXAMPLE 3

Preparation of Gelatin Capsules Containing 8% Methylphenidate and 20% Citric Acid.

Composition (for 1000 capsules)

| | |
|---|---|
| dl-methylphenidate | 20.0 gm |
| microcrystalline cellulose | 88.0 gm |
| modified starch | 88.0 gm |
| magnesium stearate | 4.0 gm |
| citric acid | 50.0 gm |

The microcrystalline cellulose, modified starch, and di-methylphenidate are granulated with water and then passed through a 0.9 mm mesh screen and dried at 500° C. The dried material is passed through a 0.9 mm mesh screen and blended with the magnesium stearate and citric acid. The resulting blend is encapsulated using size #1 hard shell gelatin capsule.

### EXAMPLE 4

Studies of Irritation Activity.

A tablet prepared in Example 1 is placed on a glass plate and crushed to form a powder. Four human subjects receive a needle prick on the middle finger. After one minute, the powder from one complete tablet is applied by means of a dropper to the finger in the area of the needle prick to each subject individually. A fourth subject receives a powder applied in the area of the needle prick which was prepared according to the procedure in Example 1 except without citric acid. The subjects are not told whether or not they received the placebo. The subjects rate the degree of irritation according to the following scale. Hypothetical test results are summarized in Table I.

Description of Irritation

NO Irritation

SLIGHT Irritation—(Barely perceptible stinging, burning or itching)

MILD Irritation—(Definite stinging, burning or itching)

MODERATE Irritation—(Distinctly uncomfortable stinging, burning or itching, constantly aware of irritation)

SEVERE Irritation—(Continuous stinging, burning or itching, and intensely uncomfortable)

TABLE I
Hypothetical Test Results

| | Type of | Amount of | Description of |
|---|---|---|---|
| Subject | Irritant | Irritant | Irritant |
| A | None | None | None |
| B | Citric Acid | 10% | Mild |
| C | Citric Acid | 10% | Slight |
| D | Citric Acid | 10% | Slight |

### EXAMPLE 5

Studies of Irritation Activity.

A tablet prepared in Example 2 is placed on a glass plate and crushed to form a powder. Four human subjects receive a needle prick on the middle finger. After one minute, the powder from one complete tablet is applied by means of a dropper to the finger in the area of the needle prick to each subject individually. A fourth subject receives a powder applied in the area of the needle prick which was prepared according to the procedure in Example 2 except without citric acid. The subjects are not told whether or not they received the placebo. The subjects rate the degree of irritation according to the following scale. Hypothetical test results are summarized in Table II.

TABLE II
Hypothetical Test Results

| | Type of | Amount of | Description of |
|---|---|---|---|
| Subject | Irritant | Irritant | Irritant |
| A | None | None | Moderate |
| B | Citric Acid | 16% | Moderate |
| C | Citric Acid | 16% | Mild |
| D | Citric Acid | 16% | None |

### EXAMPLE 6

Studies of Irritation Activity.

A capsule prepared in Example 3 is placed on a glass plate and crushed to form a powder. Four human subjects receive a needle prick on the middle finger. After one minute, the powder from one complete capsule is applied by means of a dropper to the finger in the area of the needle prick to each subject individually. A fourth subject receives a powder applied in the area of the needle prick which was prepared according to the procedure in Example 3 except without citric acid. The subjects are not told whether or not they received the placebo. The subjects rate the degree of irritation according to the following scale. Hypothetical test results are summarized in Table III.

TABLE III
Hypothetical Test Results

| | Type of | Amount of | Description of |
|---|---|---|---|
| Subject | Irritant | Irritant | Irritant |
| A | None | None | Moderate |
| B | Citric Acid | 20% | Severe |
| C | Citric Acid | 20% | None |
| D | Citric Acid | 20% | Severe |

The results in Tables I, II and III clearly show that the compositions of the present invention produce irritation when contacted with dermis layer of skin or mucous membrane, while the compositions prepared without an irritant do not produce any irritation to the dermis layer of skin. The results also show that the degree of irritation is correlated with the amount of mucous membrane irritant in the composition.

The present invention is based on the discovery that a central nervous system stimulant such as methylphenidate in combination with a mucous membrane irritant reduces or eliminates potential drug abuse by producing “irritation” when contacted with the dermis layer of skin or mucous membrane, and thus, prevents nasal absorption and/or injectability of the drug.

While the invention has been described with particular reference to certain embodiments thereof, it will be understood that changes and modifications may be made by those of ordinary skill within the scope and spirit of the following claims:

What is claimed is:

1. A pharmaceutical composition which reduces or eliminates the drug abuse potential of central nervous system stimulant comprising:
(a) a central nervous system stimulant selected from the group consisting of methylphenidate, amphetamine, methamphetamine, and combinations thereof; and
(b) a mucous membrane irritant selected from the group consisting of organic and inorganic acid, salt, ketone, nitrite, sulfide, bisulfate, persulfate, glycerophosphate, hypophosphate, borate, titanate, amino acid, peptide, and combinations thereof, wherein the mucous membrane irritant produces irritation when contacted with the dermis layer of skin or mucous membrane.

2. A pharmaceutical composition which reduces or eliminates the drug abuse potential of central nervous system stimulant comprising:
(a) a central nervous system stimulant selected from the group consisting of methylphenidate, amphetamine, methamphetamine, and combinations thereof; and
(b) a mucous membrane irritant selected from the group consisting of mono-, di-, tri-, and tetra-carboxylic acids, and combinations thereof, wherein the mucous membrane irritant produces irritation when contacted with the dermis layer of skin or mucous membrane.

3. The composition according to claim 1 wherein the mucous membrane irritant is selected from the group consisting of anthralin, camphor, canthariden, capsicum, coal tar, ichthammol, juniper tar, menthol, Peruvian balsam, pine tar, aluminum chloride, resorcinol, storax, tolu balsam, nitric acid, phenol, podofilox, podophyllum, potassium hydroxide, silver nitrate, trichloroacetic acid, benzoyl peroxide, fluorouracil, salicylic acid, retinoic acid, ethanol, isopropanol, selenium sulfide, benzalkonium chloride, allantoin, aminobenzoic acid, propenoic acid, dihydroxyacetone, dioxybenzone, octyl methoxycinnamate, 2,4,6,8-nonanetetraenoic acid, homosalate, hydrogen peroxide, hydroxyurea, citric acid, lactic acid, glycolic acid, salicylic acid, pyromellitic acid, pyromellitic dianhydride, pyruvic acid, acetic acid, acrylic acid, trichloroacetic acid, 1-pyrrolidone-5-carboxylic acid, capryloyl salicylic acid, hydroxy decanoic acid, hydroxy octanoic acid, gluconolactone, methoxypropyl gluconamide, malic acid, maleic acid, tartaric acid, mandelic acid, gluconic acid, sodium chloride, ethylenediaminetetraacetic acid disodium salt, sodium boroformate, sodium bicarbonate, and dipropyl ketone.

4. The composition according to claim 3 wherein the mucous membrane irritant is selected from the group consisting of citric acid, lactic acid, glycolic acid, salicylic acid, pyromellitic acid, pyromellitic dianhydride, pyruvic acid, acetic acid, acrylic acid, trichloroacetic acid, 1-pyrrolidone-5-carboxylic acid, capryloyl salicylic acid, hydroxy decanoic acid, hydroxy octanoic acid, gluconolactone, methoxypropyl gluconamide, malic acid, maleic acid, tartaric acid, mandelic acid, and gluconic acid.

5. The composition according to claim 4 wherein the mucous membrane irritant is citric acid.

6. The composition according to claim 1 wherein the central nervous system stimulant is present in an amount of from about 0.1 to about 90 weight percent, based on the total weight of the composition.

7. The composition according to claim 6 wherein the central nervous system stimulant is present in an amount of from about 1 to about 50 weight percent, based on the total weight of the composition.

8. The composition according to claim 7 wherein the central nervous system stimulant is present in an amount of from about 2 to about 10 weight percent, based on the total weight of the composition.

9. The composition according to claim 1 wherein the mucous membrane irritant is present in an amount of from about 0.1 to about 60 weight percent, based on the total weight of the composition.

10. The composition according to claim 9 wherein the mucous membrane irritant is present in an amount of from about 1 to about 40 weight percent, based on the total weight of the composition.

11. The composition according to claim 10 wherein the mucous membrane irritant is present in an amount of from about 5 to about 20 weight percent, based on the total weight of the composition.

12. The composition according to claim 1 which is in a form selected from the group consisting of powder, granules, solution, suspension, emulsion, and combinations thereof.

13. The composition according to claim 12 which is in a form of a solid.

14. The composition according to claim 12 wherein the composition is administered in a form selected from the group consisting of a capsule, cachet, and tablet.
